(19) United States
(12) Patent Application Publication
(10) Pub. No.: US 2014/0056965 A1
(43) Pub. Date: Feb. 27, 2014

(54) METHOD AND SYSTEM FOR TREATING DEPRESSION OR EPILEPSY

(71) Applicant: BOSTON SCIENTIFIC NEUROMODULATION CORPORATION, Valencia, CA (US)

(72) Inventor: Kerry Bradley, Glendale, CA (US)

(73) Assignee: BOSTON SCIENTIFIC NEUROMODULATION CORPORATION, Valencia, CA (US)

(21) Appl. No.: 13/970,449

(22) Filed: Aug. 19, 2013

Related U.S. Application Data
(60) Provisional application No. 61/693,054, filed on Aug. 24, 2012.

(51) Int. Cl. A61K 9/70; A61K 31/167; A61K 31/045

(52) U.S. Cl. CPC A61K 9/7023; A61K 31/045; A61K 31/167
USPC 424/449; 514/729; 514/626

(57) ABSTRACT

A removable patch configured to be applied over a skin surface, comprises an adhesive layer disposed on the removable patch, and a delivery layer disposed on the removable patch. The delivery layer carries an activating chemical neuromodulator adapted to be transcutaneously applied to afferent A fibers disposed under the skin surface. The delivery layer further carries an inhibiting chemical neuromodulator adapted to be transcutaneously applied to C fibers disposed under the skin surface. A method for treating a patient suffering from a neurological disorder comprises transcutaneously applying an activating chemical neuromodulator over a skin surface adjacent to a trigeminal nerve tissue of the patient, and activating afferent A fibers in the trigeminal nerve, the activation being carried out by the chemical neuromodulator.

### RELATED APPLICATION DATA

The present application claims the benefit under 35 U.S.C. §119 to U.S. provisional patent application Ser. No. 61/693,054, filed Aug. 24, 2012. The foregoing application is hereby incorporated by reference into the present application in its entirety.

### FIELD OF THE INVENTION

The present invention generally relates to neuromodulator methods and systems, and more particularly relates to chemical neuromodulator systems and methods for treating epilepsy, movement disorders, and similar indications.

### BACKGROUND OF THE INVENTION

Epilepsy is characterized by a tendency to recurrent seizures that can lead to loss of awareness, loss of consciousness, and/or disturbances of movement, autonomic function, sensation (including vision, hearing and taste), mood, and/or mental function. Epilepsy afflicts 1-2% of the population in the developed world. The mean prevalence of active epilepsy (i.e., continuing seizures or the need for treatment) in developed and undeveloped countries combined is estimated to be 7 per 1,000 of the general population, or approximately 40 million people worldwide. Studies in developed countries suggest an annual incidence of epilepsy of approximately 50 per 100,000 of the general population. However, studies in developing countries suggest this figure is nearly double at 100 per 100,000.

Epilepsy is often, but not always, the result of underlying brain disease. Any type of brain disease can cause epilepsy, but not all patients with the same brain pathology will develop epilepsy. The cause of epilepsy cannot be determined in a number of patients; however, the most commonly accepted theory posits that it is the result of an imbalance of certain chemicals in the brain, e.g., neurotransmitters, and/or abnormal structural defects in the brain (tumor, lesions from past injury) which can alter brain signaling. Children and adolescents are more likely to have epilepsy of unknown or genetic origin. The older the patient, the more likely it is that the cause is an underlying brain disease such as a brain tumor or cerebrovascular disease.

Trauma and brain infection can cause epilepsy at any age, and in particular, account for the higher incidence rate in developing countries. For example, in Latin America, neurocysticercosis (cysts on the brain caused by tapeworm infection) is a common cause of epilepsy; in Africa, AIDS and its related infections, malaria and meningitis, are common causes; in India, AIDS, neurocysticercosis and tuberculosis, are common causes. Febrile illness of any kind, whether or not it involves the brain, can trigger seizures in vulnerable young children, which seizures are called febrile convulsions. About 5% of such children go on to develop epilepsy later in life. Furthermore, for any brain disease, only a proportion of sufferers will experience seizures as a symptom of that disease. It is, therefore, suspected that those who do experience such symptomatic seizures are more vulnerable for similar biochemical/neurotransmitter and structural reasons.

In addition to epilepsy, post-traumatic stress disorder (PTSD) in patients is one of the most commonly occurring of anxiety disorders, including treatments that cause immense economic burden. In general, PTSD follows a psychologically disturbing and traumatic event, such as terrorist incidents, war situations, sexual abuse, physical abuse, accidents, sport injuries, natural disasters, and the like, after which a patient may suffer from a mental condition of fear, helplessness, anger, etc. PTSDs occurring therefore can be severe, chronic, and widely prevalent, with some studies suggesting a lifetime prevalence of 1.3 to 7.8 in general population. Certain cases of PTSD may include persistence of depression related symptoms which may worsen with the passage of time, preventing the patient from returning to a normal lifestyle. More particularly, a considerable percentage of population suffering from PTSD may recover over substantially long periods, causing mental and financial stress to the patient's immediate family members and caretakers as well.

Furthermore, major depression in men, women, and young adults, have been observed over the years as well. Such form of a neurological disorder results in reduced ability to engage in productive work and interpersonal relationships. According to the American Psychiatric Association, around 5-9% of women and between 2-3% of men meet the diagnostic criteria for major depression at any period. In addition, it has been observed that 10-25% of women and 5-10% of men suffer from major depression at least once in their lifetimes. Usually, major depression is characterized by its sharp contrast to usual functioning. Upon observation, a person with major depression may behave normally, but may suddenly develop symptoms of severe depression over a period. Accordingly, major depression is a health problem that poses a tremendous challenge and financial burden on the suffering individuals and their guardians. Individuals suffering from such disorders are not able to enjoy everyday life because of feelings of extreme hopelessness and worthlessness. Moreover, there is an increasing amount of suicides noted among such individuals as well.

Trigeminal Nerve Stimulation (TNS) is a new investigational treatment of brain disorders, such as epilepsy, post-traumatic stress disorder (PTSD), and major depression. Such a treatment involves placing electrical pads or electrodes over a facial region or on the forehead over branches of the trigeminal nerves and supplying low-amplitude electrical pulses to the electrical pads from a neurostimulator (typically externally worn on the patient, such as the belt) via wires routed underneath the clothing from the neurostimulator to the electrical pads. These treatments ameliorate neurological ailments such as depression. In particular, this process is valuable for its simplicity and comfort, but it does require electrical power sources for activating the electrodes. Techniques and systems for delivering neurostimulation to a patient's trigeminal nerve are disclosed in, for example, U.S. Pat. App. Pub. No. 2011/0009920, which is expressly incorporated herein by reference.

Thus, there exists a need to provide simple and effective means for treating epilepsy, post-traumatic stress disorder (PTSD), and similar such neurological disorders.

### SUMMARY OF THE INVENTION

In accordance with one aspect of the present inventions, a removable patch configured to be applied over a skin surface is provided. The removable patch comprises an adhesive layer disposed on the removable patch, and a delivery layer disposed on the removable patch. The delivery layer carries an activating chemical neuromodulator (e.g., menthol) adapted to be transcutaneously applied to afferent A fibers disposed under the skin surface. The delivery layer further carries an inhibiting chemical neuromodulator (e.g., lidocaine) adapted to be transcutaneously applied to C fibers disposed under the skin surface.

In accordance with another aspect of the present inventions, a method for treating a patient suffering from a neurological disorder (e.g., epilepsy, depression, or post-traumatic stress disorder) is provided. The method comprises transcutaneously applying an activating chemical neuromodulator (e.g., menthol) over a skin surface (e.g., skin on a human face, such as over a junction of zygomatic and maxilla bones of a skull, or on the supraorbital glabrous forehead) adjacent to a trigeminal nerve tissue of the patient, and activating afferent A fibers in the trigeminal nerve, the activation being carried out by the chemical neuromodulator.

The activating chemical neuromodulator may be carried in a removable patch (which may be translucent or transparent), in which case, the method may further comprising adhering the removable patch to the skin surface adjacent the trigeminal nerve tissue. An optional method further comprises transcutaneously applying an inhibiting chemical neuromodulator (e.g., lidocaine) over the skin surface adjacent to the trigeminal nerve tissue of the patient, and inhibiting C fibers in the trigeminal nerve, the inhibiting being carried out by the inhibiting chemical neuromodulator. Another optional method further comprises transcutaneously applying a vasoconstricting chemical over the skin surface adjacent to the trigeminal nerve tissue of the patient, and constricting blood vessels adjacent to the trigeminal nerve tissue, the constricting being carried out by the vasoconstricting chemical, thereby increasing the time that the activating chemical neuromodulator is in the trigeminal nerve tissue.

Other and further aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present invention are obtained, a more particular description of the present invention briefly described above will be rendered by reference to specific embodiments thereof, which are illustrated in the accompanying drawings.

Understanding that these drawings depict only typical embodiments of the invention and are not therefore to be considered limiting of its scope, the invention will be described and explained with additional specificity and detail through the use of the accompanying drawings in which:

FIG. 1 is a schematic of trigeminal nerves disposed under a facial structure of the human body;

FIG. 2 is an isometric view of an exemplary embodiment of a transdermal patch constructed in accordance with the present inventions;

FIG. 3 is a sectional view of the transdermal patch of FIG. 2;

FIG. 4 is an exemplary application of the transdermal patch of FIG. 2 on the facial structure of FIG. 1; and

FIG. 5 is another exemplary application of the transdermal patch of FIG. 2 on the facial structure of FIG. 1.

### DETAILED DESCRIPTION

The present disclosure provides a drug delivery system for treating medical diseases such as epilepsy, PTSD, and forms of depression, by neuromodulating the trigeminal nerve. The trigeminal nerve includes a number of branches of sensory nerves which are responsible for sensation in the face, as well as certain motor functions such as biting, chewing, and swallowing.

With reference to FIG. 1, a patient 100 includes a trigeminal nerve 102 underlying the patient's face 108. The trigeminal nerve 102 contains both afferent and efferent components. The afferent portions of trigeminal nerve 102 provide tactile, proprioceptive, and nociceptive afference of the face and mouth. Efferent fibers connect the trigeminal nerve 102 to subcutaneous regions, providing motor functions such as biting, chewing, and swallowing. As would be known in the art, the patient's face 108 also includes a maxilla region 106, a zygomatic region 104, and a supraorbital forehead 112 overlying the frontal bone of the skull.

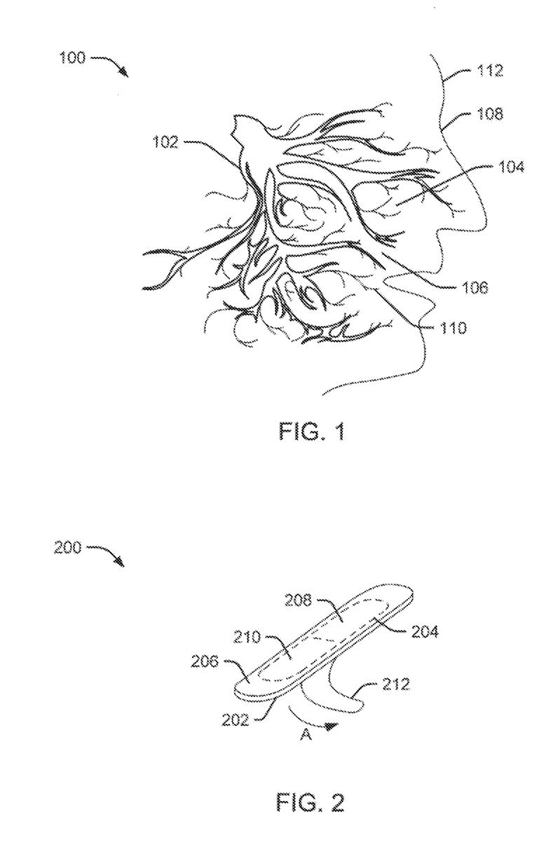

With reference to FIG. 2, a removable transdermal patch 200 constructed according to an embodiment of the present invention will now be described. The removable patch 200 may be adapted to be applied over the human skin, and more specifically, to the patient's face 108. The removable patch 200 may be sized and shaped appropriately for human use and may thus be configured to be removable. In particular, the removable patch 200 may include an adhesive portion 202 and a delivery layer 204, the details of which are discussed further in the application.

The removable patch 200 is a generally flexible, flat strip of material, adapted to be applied to the patient's face 108 for the transcutaneous application of medication. It may be adequately elastic so that its application over the patient's face 108 may suitably accommodate movements of the jaw during motor functions such as biting and chewing, along with movements of the skin in relation to the bone and movements between the regions of the maxilla and zygomatic regions of the skull, etc. Further, the removable patch 200 may be translucent or transparent, allowing it to be practically invisible in social situations, and thereby enabling it to be worn in public during Activities of Daily Living (ADL). In some embodiments, the removable patch 200 may vary in size, shape, design, pattern, and style that may appeal to a user. For example, figures of animated characters over surface of the removable patch 200 may appeal to and may encourage children to substantially prolong the patch's usage.

The removable patch 200 includes a body portion 206 as well as a disposable portion 212. The body portion 206 is sufficiently resistant to inadvertent contact with fluids such as water, perspiration, and the like, allowing continued use over prolonged periods. Further, the body portion 206 may be adequately ventilated, including a number of pores in its structure. The pores may enable passage of ambient air into the region where the removable patch 200 is applied thereby avoiding excessive perspiration, or sensations of itching, etc., to a patient. In some embodiments the pores are sufficiently large that they could be termed apertures. In common with other devices featuring an adhesive for application to the skin, the removable patch 200, including the disposable portion 212 applied to its underside, protects the adhesive portion 202. This member may be simply peeled off and discarded immediately before application of the removable patch 200 to the patient.

The adhesive portion 202 allows the removable patch 200 to be applied to and adhere to a patient's skin, particularly to the patient's face 108. This layer forms the underside of the removable patch 200, and it includes an adhesive material adapted to adhere to the human skin. The adhesive material may be skin-friendly—that is, a material that does not irritate the skin, permitting the patient to wear it for multiple hours without itching or other undesirable effects. More explicitly, the adhesive portion 202 may include conventional materials or any suitable bio-compatible hydrophilic adhesive such as resin emulsion adhesives. For example, an acrylate emulsion adhesive or a co-polymer of vinyl acetate and dioctyl maleate may be applied. Of these, acrylic emulsion adhesives have been observed to provide the most desired results according to the related requirements during an application of the removable patch 200. Certain hydrophilic adhesives can be used to include an acrylic ester co-polymer and a vinyl acetate resin as well.

Moreover, adhesive bandages, in the form of devices to close and protect wounds, as well as conventional transdermal patches, have gained wide acceptance among patients and medical practitioners, and the adhesive portion 202 can be provided in much the same form as employed in such devices. The adhesive portion 202 may include a property to irritate the skin during prolonged application periods, encouraging the patient to remove the removable patch 200 and apply a new one as and when required.

The delivery layer 204 carries a suitable chemical neuromodulator portion, referred to as the chemical neuromodulator 208, formulated to be transcutaneously applied to afferent A fibers of the patient's trigeminal nerve 102, disposed under the skin surface. This portion may be formed of cellular foam or similar matrix material. The chemical neuromodulator 208 is more particularly an activating neuromodulator, as it functions to activate the afferent A fibers, neuromodulating the trigeminal nerve 102, thereby increasing traffic to brainstem structures (e.g., trigeminocervical nucleus), which ultimately affect higher centers in the brain and treat the neurological disorder. In some embodiments, the chemical neuromodulator 208 may include menthol or similar active agent.

In other embodiments, the employment of menthol may be replaced by or supplemented with other compounds applicable in treating neurological disorders, as known to those skilled in the art. In some embodiments, the delivery layer 204 may include multiple drugs, allowing for broadly based fiber recruitment in the patient's subcutaneous region. Accordingly, along with the activating chemical neuromodulator 208, the constituents of the delivery layer 204 may include an inhibiting chemical neuromodulator 210, such as lidocaine with epinephrine, which may have similar transfusion properties as the activating chemical neuromodulator 208. In particular, the inhibiting chemical neuromodulator 210 may be adapted to be transcutaneously applied to C fibers disposed under the skin surface. The inhibiting chemical neuromodulator 210 may be configured for a more superficial transcutaneous penetration, inhibiting at least one of C fibers, thereby helping prevent uncomfortable or noxious sensations to the patient 100. Optionally, the delivery layer 204 may include a vasoconstricting chemical, such as epinephrine or similar drugs, which causes local vasoconstriction, and thus increases the length of time that the drug is present in the tissue near the patch and is not carried away by diffusive blood flow via the vessels in the nearby tissue.

The removable patch 200 may be made of biocompatible materials, thereby preventing the development of skin irritations, allergies, and the like, in patients. The material used to manufacture the removable patch 200 may also be chemically stable, providing a long shelf life during distribution. In particular, chemical stability may enable the removable patch 200 to remain unresponsive and non-reactive to the constituents of the removable patch 200, or the delivery layer 204, disposed within the removable patch 200 at all times.

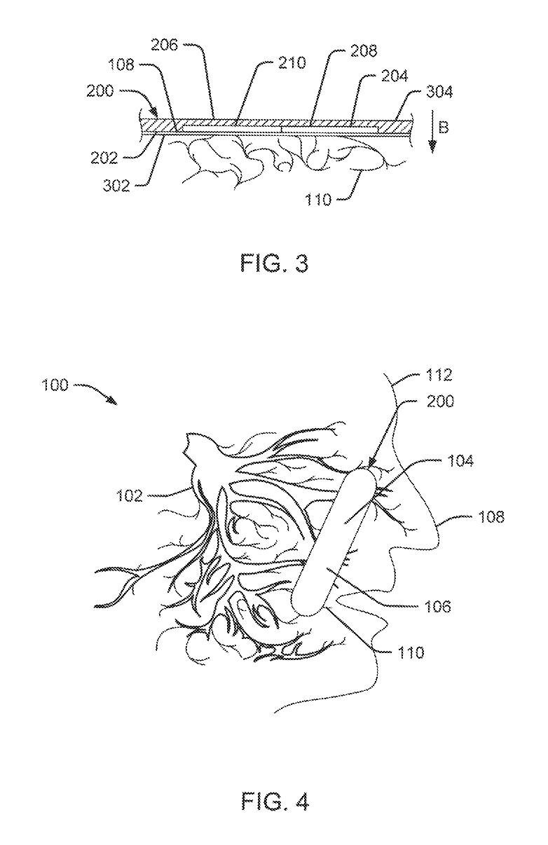

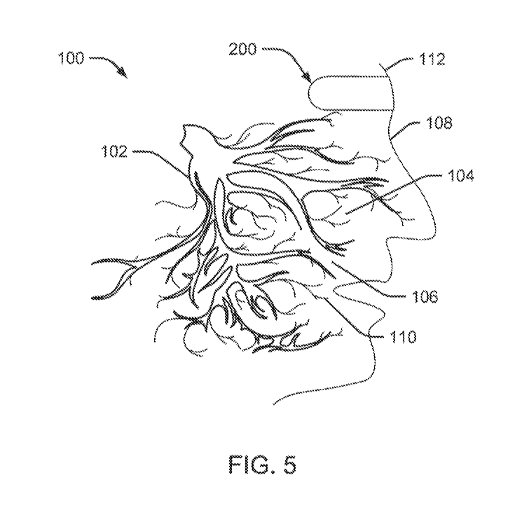

Referring now to FIGS. 3 and 4, the use of the removable patch 200 to treat a neurological or a brain disorder, such as epilepsy, depression, PTSD, in a patient will be described. In operation, the cutaneous portion or the delivery layer 204 may be placed on the patient's skin to transcutaneously deliver a specific dose of chemical neuromodulators to the trigeminal nerve 102. In an embodiment, the removable patch 200 may be placed on the patient's cheek, or over the skin surface 304 at the junction of the zygomatic region 104 and the maxilla region 106 of the skull. In another embodiment, the removable patch 200 may be placed over the supraorbital forehead 112 overlying the frontal bone of the skull, as illustrated in FIG. 5. In either event, the placement may be adjacent to a portion of the trigeminal nerve 102 to allow the removable patch 200 to neuromodulate the cutaneous branches of the trigeminal nerve 102.

The activating chemical neuromodulator 208, initially stored within delivery layer 204, passes through the adhesive portion 202 and penetrates the patient's subcutaneous regions 302 after application of the removable patch 200 to the patient's face 108. The far ends or sensory branches of trigeminal nerve afferent fibers 110 provide a path of travel, allowing the chemical neuromodulator 208 to travel deeper into the patient's tissues to reach the A fiber afferents (not shown) of the trigeminal nerve 102. Such penetration may occur after a defined period following the removable patch's application to the patient's face 108. While in application, the removable patch 200 delivers the chemical neuromodulator 208 to the afferent fibers 110 in subcutaneous regions 302. The chemical neuromodulator 208 may be transdermally diffused to the patient from the epidermal layer, across the patient's dermal layers, to the trigeminal nerve 102, as shown by arrow B. More particularly, the removable patch 200 may include a slow release formulation of the chemical neuromodulator 208, which in turn is adapted to penetrate a skin surface 304 to subcutaneous levels, allowing for at least 6 to 12 hours of drug diffusion into the patient's bloodstream. In preferred embodiments, the drug delivered provides the patient 100 with innocuous sensations, activating the afferents A fibers of the trigeminal nerve 102.

Accordingly, upon a similar application, the inhibiting chemical neuromodulator 210 initially stored within the delivery layer 204 passes through the adhesive portion 202 and penetrates the patient's subcutaneous regions 302 after application of the removable patch 200 to the patient's face 108. The inhibiting chemical neuromodulator 210 transcutaneously penetrates to reach the ends of afferent fibers 110. Traveling deeper, the inhibiting neuromodulator may reach the C fibers, where the inhibiting neuromodulator may act to block activation of the trigeminal nerve C fibers.

The optional vasoconstricting chemical initially stored within the delivery layer 204 passes through the adhesive portion 202 and penetrates the patient's subcutaneous regions 302 after application of the removable patch 200 to the patient's face 108. The vasoconstricting chemical causes local vasoconstriction, thereby increasing the length of time that the drug is present in the tissue.

An advantage of the removable patch 200 for drug delivery over other types of drug delivery such as intravenous, topical, oral, topical, intramuscular, and so forth, is that the patch provides a controlled release of the drug into the patient's body, usually through either a porous membrane covering a reservoir of neuromodulator or through body heat melting thin layers of drug embedded in the adhesive layer. Moreover, the slow release formulation, allowing for at least six and preferably twelve hours of drug elution can be categorized as another advantage of the removable patch 200.

Although particular embodiments of the present inventions have been shown and described, it will be understood that such embodiments are not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

What is claimed is:

1. A removable patch configured to be applied over a skin surface, comprising:
an adhesive layer disposed on the removable patch; and
a delivery layer disposed on the removable patch, the delivery layer carrying an activating chemical neuromodulator adapted to be transcutaneously applied to afferent A fibers disposed under the skin surface, the delivery layer further carrying an inhibiting chemical neuromodulator adapted to be transcutaneously applied to C fibers disposed under the skin surface.

2. The removable patch of claim 1, wherein the activating chemical neuromodulator is menthol.

3. The removable patch of claim 1, wherein the inhibiting chemical neuromodulator is lidocaine.

4. The removable patch of claim 1, wherein the delivery layer further carries a vasoconstricting chemical adapted to be transcutaneously applied to blood vessels disposed under the skin surface.

5. A method for treating a patient suffering from a neurological disorder, the method comprising:
transcutaneously applying an activating chemical neuromodulator over a skin surface adjacent to a trigeminal nerve tissue of the patient; and
activating afferent A fibers in the trigeminal nerve, the activation being carried out by the chemical neuromodulator.

6. The method of claim 5, wherein the activating chemical neuromodulator is menthol.

7. The method of claim 5, wherein the activating chemical neuromodulator is carried in a removable patch, the method further comprising adhering the removable patch to the skin surface adjacent the trigeminal nerve tissue.

8. The method of claim 6, wherein the removable patch is either one of translucent or transparent.

9. The method of claim 5, further comprising:
transcutaneously applying an inhibiting chemical neuromodulator over the skin surface adjacent to the trigeminal nerve tissue of the patient; and
inhibiting C fibers in the trigeminal nerve, the inhibiting being carried out by the inhibiting chemical neuromodulator.

10. The method of claim 8, wherein the inhibiting chemical neuromodulator is lidocaine.

11. The method of claim 5, further comprising:
transcutaneously applying a vasoconstricting chemical over the skin surface adjacent to the trigeminal nerve tissue of the patient; and
constricting blood vessels adjacent to the trigeminal nerve tissue, the constricting being carried out by the vasoconstricting chemical, thereby increasing the time that the activating chemical neuromodulator is in the trigeminal nerve tissue.

12. The method of claim 5, wherein the skin surface is located on a human face.

13. The method of claim 5, wherein the skin surface is located over a junction of zygomatic and maxilla bones of a skull.

14. The method of claim 5, wherein the skin surface is located on a supraorbital forehead overlying the frontal bone of a skull.

15. The method of claim 5, wherein the neurological disorder is epilepsy.

16. The method of claim 5, wherein the neurological disorder is depression.

17. The method of claim 5, wherein the neurological disorder is post-traumatic stress disorder.
